(19) United States
(12) Patent Application Publication
(10) Pub. No.: US 2007/0255298 A1
(43) Pub. Date: Nov. 1, 2007

(54) Disposable safety scalpel with reusable handle

(76) Inventors: Ilija Djordjevic, East Granby, CT (US); Sushil K. Kanwar, West Hartford, CT (US)

Correspondence Address: ALIX YALE & RISTAS LLP, 750 MAIN STREET, SUITE 1400, HARTFORD, CT, 06103, US

(21) Appl. No.: 11/796,957

(22) Filed: Apr. 30, 2007

Related U.S. Application Data
(60) Provisional application No. 60/796,807, filed on May 1, 2006.

(51) Int. Cl. A61B 17/32

(52) U.S. Cl. 606/167

(57) ABSTRACT

A blade assembly for a surgical scalpel that includes a blade carrier with a blade attached one end thereof. A protective cover covers the blade and blade holder. The blade carrier has a detent and the protective cover has a latch in releasable engagement with the detent to hold the protective covering in a protecting position covering said blade assembly and said blade. Also a surgical scalpel is disclosed which comprises a handle having two prongs extending from one end thereof, one of said prongs being resilient, the other of said prongs having a latching tooth at its distal end, A disposable blade assembly includes a blade carrier having a surgical blade attached thereto. The blade carrier has a surface defining a window with a shoulder into which said prongs extend with said latching tooth engaging said shoulder when said handle is inserted into said blade carrier. A protective cover covers the blade carrier and is moveable between a covered position covering the blade and a cutting position in which the blade is exposed for use.

### CROSS REFERENCE TO RELATED APPLICATION

This application claims the priority of U.S. patent application Ser. No. 60/796,807 filed May 1, 2006, the disclosure of which is hereby incorporated by reference.

### TECHNICAL FIELD

The present invention relates generally to surgical scalpels, and in particular, to scalpels having a reusable handle and a disposable blade assembly.

### BACKGROUND

Conventional surgical instruments present a significant risk of harm to surgeons, nurses and other medical personnel. The risk of getting cut or punctured by an exposed blade increases during medical emergencies when various surgical instruments are quickly passed from person to person. Exposed blades can also compromise the integrity of surgical gloves during a procedure, which may increase the risk of infection via contact with blood or other bodily fluids.

Retractable blade guards on surgical scalpels are well known. However, there remains a need for a scalpel and disposable blade assembly that guards against inadvertent injury yet can be assembled quickly, easily, and reliably.

### SUMMARY

According to one aspect there is provided a blade assembly for a surgical scalpel comprising a blade carrier and a blade attached to the blade carrier at one end thereof. A protective cover covering the blade and blade holder is provided. The blade carrier has a detent and the protective cover has a latch in releasable engagement with the detent to hold the protective covering in a protecting position covering the blade assembly and the blade.

According to another aspect, there is provided a surgical scalpel comprising a handle having two prongs extending from one end thereof, one of the prongs being resilient, the other of the prongs having a latching tooth at its distal end. A disposable blade assembly is provided which includes a blade carrier having a surgical blade attached thereto. The blade carrier has a surface defining a window with a shoulder into which the prongs extend with the latching tooth engaging the shoulder when the handle is inserted into the blade carrier.

According to yet another aspect there is provided a surgical scalpel comprising a handle having two prongs extending from one end thereof. One of the prongs has a latching tooth at its distal end, and the prongs are separated by a channel extending into the handle. A disposable blade assembly includes a blade carrier having a surgical blade attached thereto. The blade carrier has a surface defining a window with a shoulder into which the prongs extend with the latching tooth engaging the shoulder when the handle is inserted into the blade carrier.

According to a still further aspect there is provided a surgical scalpel comprising a disposable blade assembly having a first end and a second end defining at least one end of a mounting window. A protective sleeve is provided on the blade assembly. A reusable handle has a distal end and a proximal end defining a mounting portion that releasably engages the mounting window, wherein the mounting portion engages the mounting window with an audible click that ensures complete engagement.

It will thus be appreciated that a significantly improved combination of a handle and a disposable blade assembly is described herein. In the preferred embodiment the handle includes at least two prongs, one of which is resilient and outwardly biased. The blade assembly includes a blade, a blade carrier and a retractable protective sleeve with a dependant tooth. The retractable protective sleeve is movable over the surface of the scalpel's handle portion to selectively expose and/or cover the blade as needed. The user slides the sleeve and positions the tooth for selectively establishing and reestablishing at least two detent conditions: (1) the covered condition and (2) a cutting condition. The protective sleeve completely covers the blade for disposal, while the handle portion of the scalpel is retained for reuse. The particular configuration of the handle and the blade assembly combination provides an extra measure of security by creating an audible “click” when the blade assembly is fully mounted to the handle portion. This “click” assures the user that the blade assembly has properly engaged the handle portion and that the scalpel is ready for use. Preferably, the structure involved in completing the “clicking” configuration also ensures that the blade assembly cannot be accidentally disengaged from the handle. The scalpel and blade assembly, including the protective sleeve, can be scaled down (miniaturized) for surgical applications that require a longer, slimmer scalpel.

### BRIEF DESCRIPTION OF THE DRAWINGS

FIG. 1 is a side view, partially in section, of a surgical scalpel according to one embodiment;

FIG. 2 is a side view of the handle of the scalpel of FIG. 1;

FIG. 3 is a side view of a disposable blade assembly usable with the handle of FIG. 2;

FIG. 4 is a side view of a handle insert according to another embodiment;

FIG. 5 is a side view showing the handle insert of FIG. 4 with a plastic over-coating;

FIGS. 6a-6g show one embodiment of the attachment of the resilient prong to the handle body;

FIGS. 7a-7c show another embodiment of the attachment of the resilient prong to the handle body;

FIGS. 8a-8e show the various components of a surgical scalpel according to one embodiment;

FIGS. 9a-9e show the assembly, disassembly and operation of the scalpel;

FIGS. 10a-10b show a user engaging the protective sleeve to move it between its two positions;

FIGS. 11, 12 and 13 show various embodiments of various features of the scalpel; and

FIGS. 14a-14d show miniaturized components of the scalpel.

FIG. 15 is a partial exploded view of another embodiment of a blade carrier and protective cover.

### DETAILED DESCRIPTION OF THE INVENTION

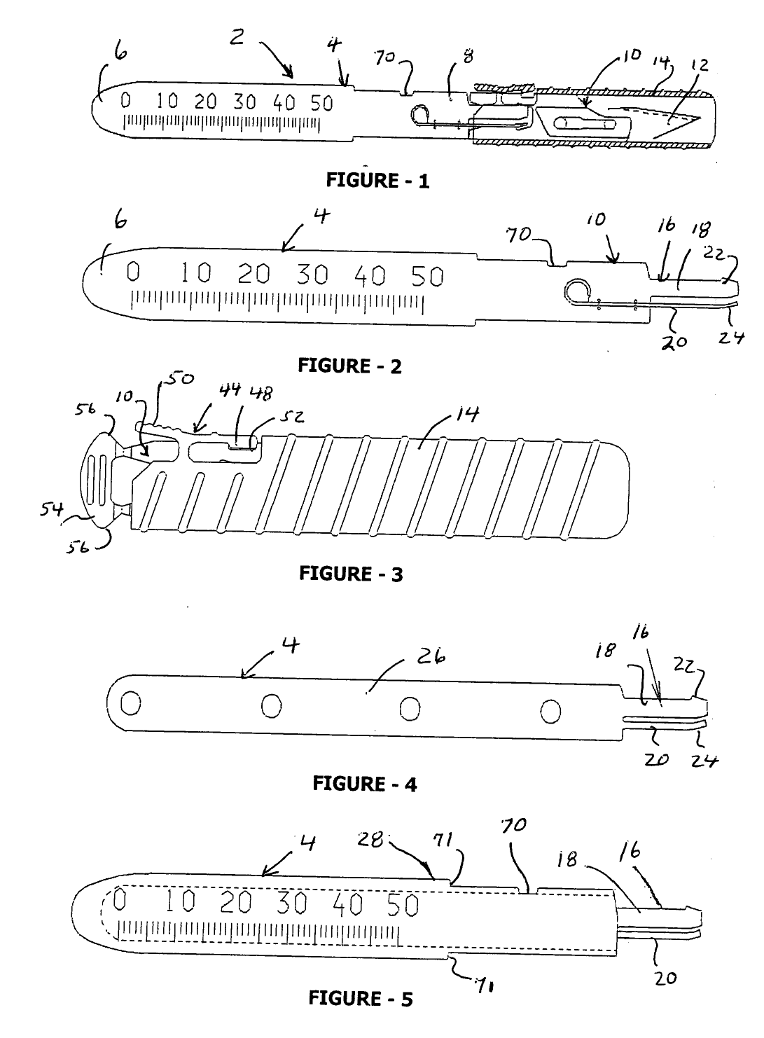

Referring to the drawings and particularly FIGS. 1-3, a scalpel 2 generally includes a handle 4 with a proximal end 6 and a distal end 8. A blade assembly 10 having a surgical blade 12 attached thereto is removeably connected to the handle 4. A protective sleeve 14 surrounds the blade assembly 10 as shown.

The distal end 8 defines the mounting portion 16 of the handle and features two mounting prongs 18 and 20. One prong 18 is rigid and is equipped with a locking tooth 22. The second prong 20 is resilient and includes a leading ramp portion 24 to facilitate insertion into the blade assembly 10. The resilient prong 20 is generally outwardly biased when the handle is inserted into the blade assembly 10. The resilient prong 20 may be added to the handle 4 as a separate component during manufacture as shown in FIG. 2. Alternatively, as shown in FIGS. 4 and 5, the handle 4 may have an inner core 26 of a suitable metal such as stainless steel where both the rigid prong 18 and resilient prong 20 are integral parts, and an outer plastic over-coating 28 that substantially encases the inner core 26.

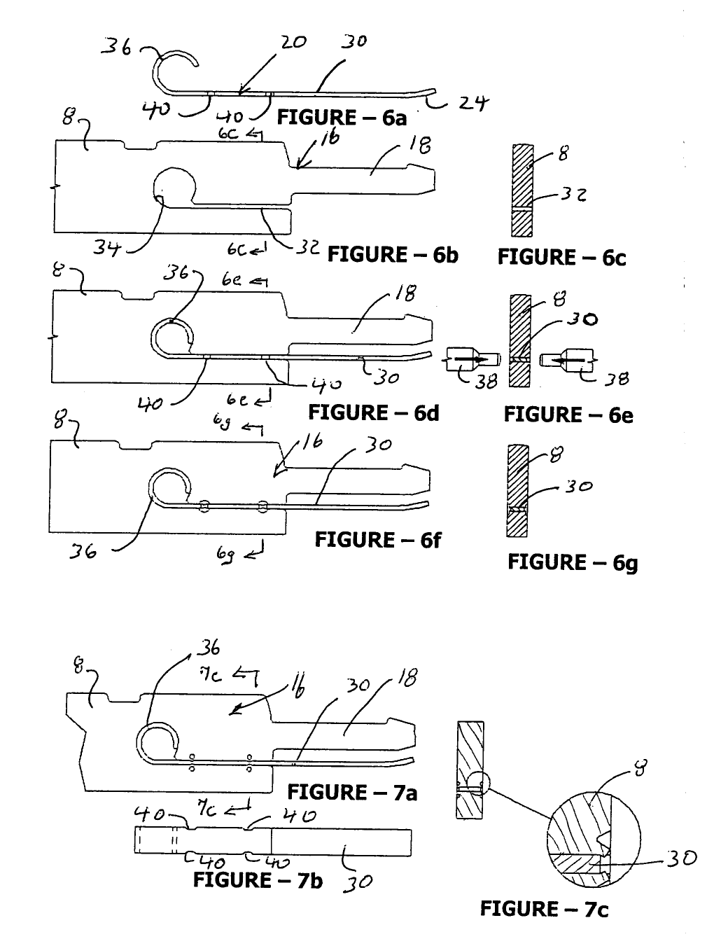

Referring to FIGS. 6a-g according to one embodiment, the resilient prong 20 may be a leaf spring 30 that is inserted into an open slot 32 in the mounting portion 16 of the handle 4 and secured. Preferably, the open slot 32 is configured as a narrow channel that terminates in a substantially circular opening 34. It is preferred that the “diameter” of the substantially circular opening 34 is smaller than the diameter of the cylindrical portion 36 of the leaf spring 30.

Once inserted in the handle 4, the leaf spring 30 may be staked or tacked in place. It is preferred that the leaf spring 30 is secured to the handle 4 using at least a pair of staking pins 38, one on each side of the handle 4 as shown in FIG. 6e. The staking pins 38 make contact with the handle 4 at a series of points along the slot 32 containing the leaf spring 30. Preferably, there is a plurality of pairs of notches or cut-outs 40 along the length of the leaf spring 30. The notches or cutouts 40 provide room for material that is displaced or deposited over the leaf spring 30 during the fastening process. It is preferred that when the staking pins 38 make contact with the handle 4, the staking pins 38 displace material into the cut-outs 40 of the leaf spring 30 while at the same time the pins 38 locally deform the handle 4 at these same locations by creating a shallow cavity. In this way, the material displaced by the pins 38 fixes the leaf spring 30 laterally in place. The ends of the staking pins 38 (i.e. the surfaces making contact with the handle) may be either cylindrical or tapered.

FIGS. 6a-f illustrate the embodiment of the staking procedure where the leaf spring 30 is secured to the handle 4 using cylindrical staking pins 38 as shown in FIG. 6d. As shown in FIG. 6e, after the leaf spring 30 is installed into the slot 32, at least two staking pins 38 are moved simultaneously in the direction indicated by the arrows. Generally, there is one staking pin 38 on each side of the handle. After the staking pins 38 make contact with the handle 4, the pins 38 displace material into the cutouts 36 in the leaf spring 30, thereby fixing the position of the leaf spring 30 laterally.

FIGS. 7a-c show the embodiment wherein the leaf spring 30 is secured to the handle 4 using tapered staking pins. The enlarged section depicted in FIG. 7c shows the details of the handle 4 in the area where the leaf spring has been staked in place. The cut outs 40 in the leaf spring 30 provide room for material to flow during the staking operation.

FIGS. 6f and 6g show the finished product of the staking process using cylindrical pins. It should be understood that each staking pin 38 has simultaneously displaced material at both upper and lower edges of the cavity and on the left and right sides of the handle surface. If the staking pins 38 are tapered, then depositing sufficient material to laterally secure the leaf spring is at least a two step process. Specifically, a tapered pin 38 must displace material on one edge of the cavity and then displace material on the other edge. Preferably, the pair of tapered staking pins 38 will displace material first at the upper edge of the cavity and then at the lower edge. One advantage to using the tapered staking pins 38 is that the axial force required to accomplish the staking is much lower than the force used when using cylindrical pins 38.

It should be understood that the method of fastening the leaf spring 30 to the handle 4 is not limited to staking as described above. Other fastening methods, such as adhesives, soldering, laser welding and EB (electron beam) welding may be used to secure the resilient prong to the handle without departing from the spirit or scope of the invention. In another embodiment, a spring made out of a round wire can be pressed and secured into a round receiving channel that runs parallel with the longitudinal axis of the handle. Preferably, the receiving channel is created by drilling.

In the embodiment shown in FIGS. 4 and 5, the handle 4 is a two-piece component having an inner core 26 and an outer plastic over-coating 28. Preferably, the inner core 26 is manufactured of stainless steel that is stamped in a configuration that is “near net shape.” In other words, the stamped inner core preferably incorporates all of the features, i.e. the notch(es), the rigid prong 18 as well as the resilient prong 20, as one piece. The handle 4 is completed via the application of a plastic over-coating 28. Preferably, the plastic over-coating 28 is molded over the inner core 26. Alternatively, the inner core 26 is inserted into a pre-made, tight-fitting, plastic sleeve. It should be understood that at least the two prongs 18 and 20 are not encased by the plastic over-coating 28 but rather the prongs 18 and 20 extend from the plastic over-coating 28 to form the mounting portion 16 of the handle.

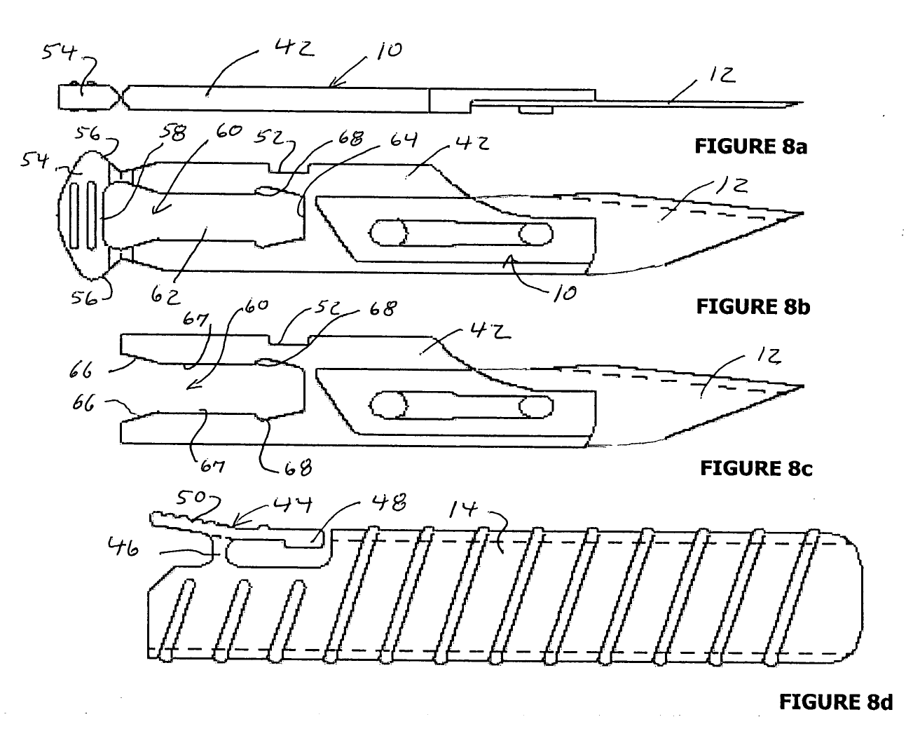

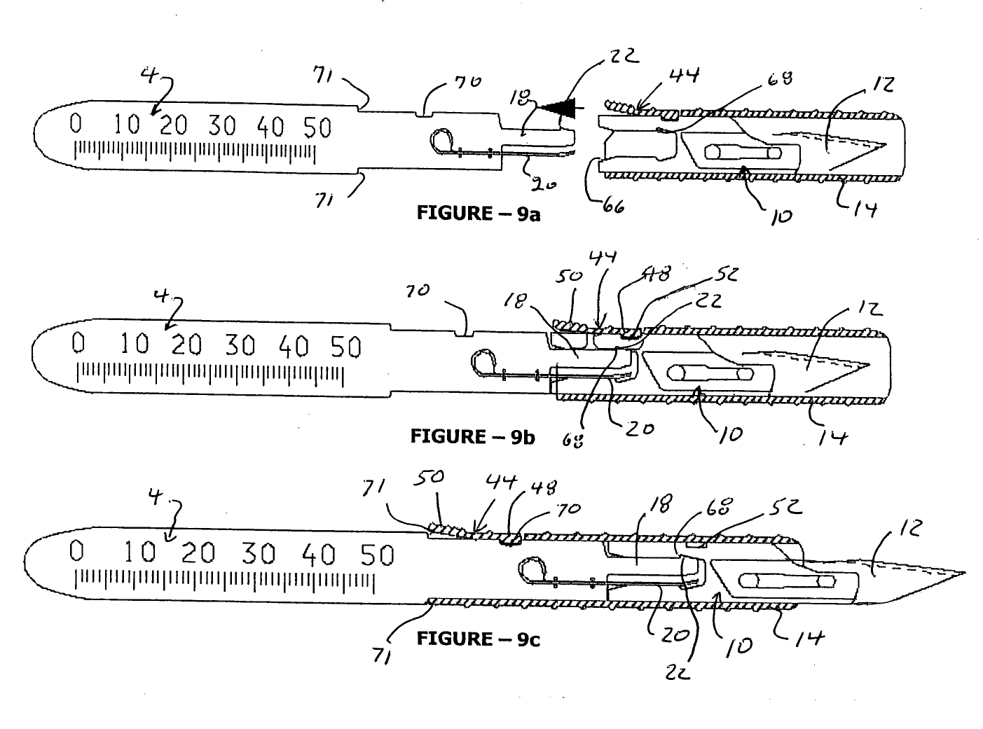

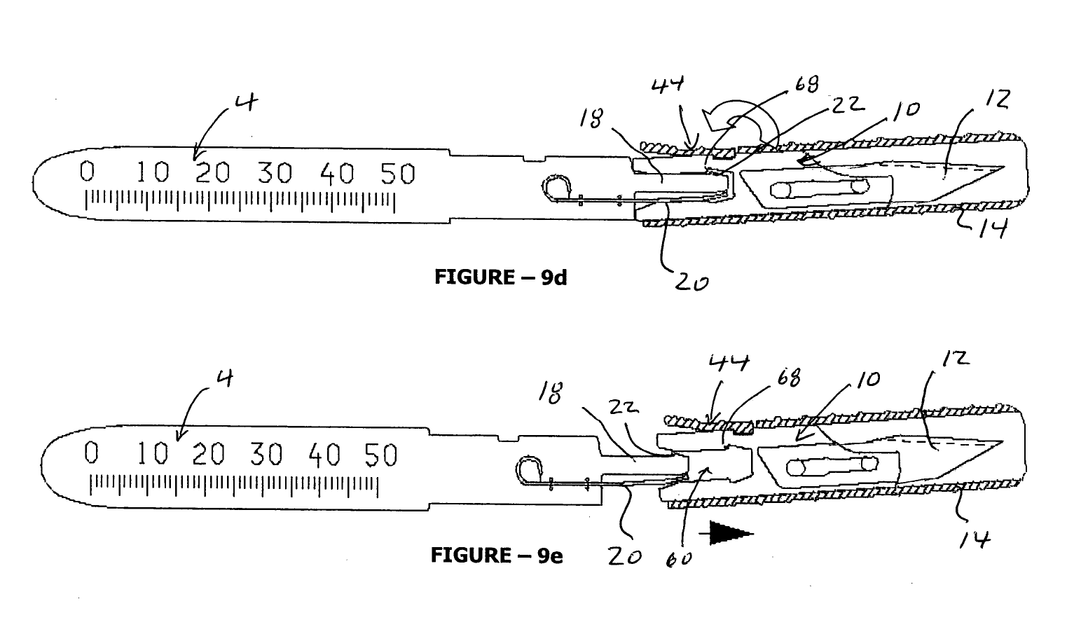

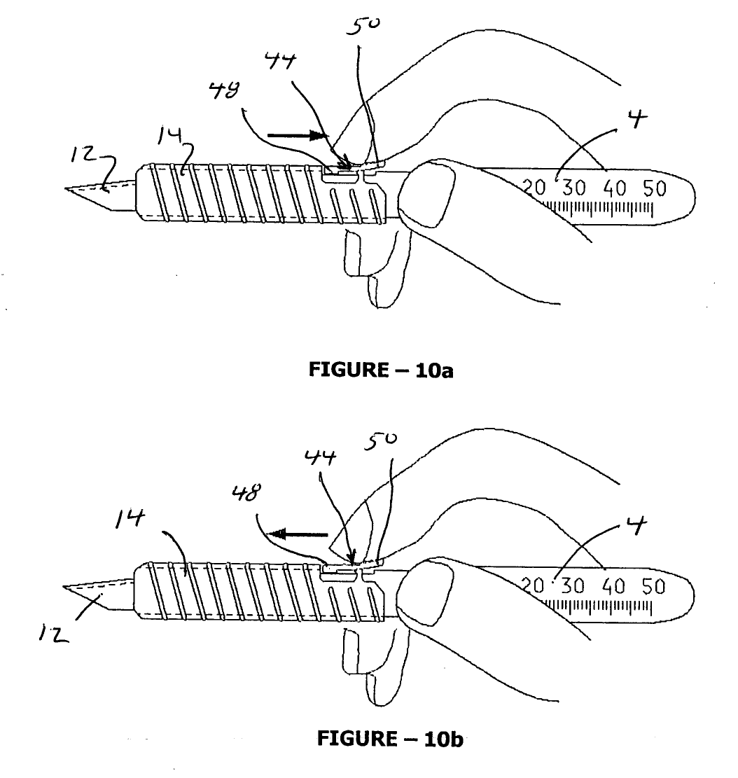

Referring to FIG. 3 and FIGS. 8a-d, the blade assembly 10 includes a blade carrier 42 to which the blade 12 is attached. In one embodiment the blade 12 is permanently fastened to the blade carrier 42 and may be of any shape or size. The protective sleeve 14 may be transparent and include an integral fulcrumed lever 44 adjacent one of its ends mounted on a pair of spaced resilient legs 46. The lever 44 has a depending tooth 48 at one end and a finger button 50 at the other end whereby depressing the finger button 50 raises the depending tooth 48. The protective sleeve is open at both its ends. The protective sleeve 14 is mounted on the blade assembly 42, and, in the covered position in which it completely covers the blade 12 as shown in FIG. 3, the depending tooth 48 releasably engages a notch 52 on the top edge of the blade carrier 42.

The blade carrier 42 also features a breakaway tab 54. The tab 54 is configured with shoulders 56 to prevent the premature removal of the protective sleeve 14 from the blade assembly 10, i.e. before the blade assembly 10 is mounted to the handle 4. Preferably, when the breakaway tab 54 is in place, the configuration of the shoulders 56 of the tab 54 makes it impossible to slide the protective sleeve 14 over the tab 54 and expose the blade 12. It is also preferred that the breakaway tab 54 forms a first end portion 58 of a mounting window 60 and must be removed in order to assemble the scalpel 2.

Initially, the mounting window 60 is an enclosed, channel-type opening with an intermediate portion 62 that extends toward the blade end of the blade carrier and terminates at a second end portion 64. The intermediate portion 62 of the mounting window 60 begins with a narrowing ramp 66 but then continues with parallel surfaces 67 spaced apart a substantially constant width. The second or inner end portion 64 of the mounting window 60 is delineated by at least one shoulder 68 that sharply increases the width of the window 60 at that point. After the shoulder 68, the width of the window may gradually decrease as shown.

To join the blade assembly 10 to the mounting portion 16 of the handle 4, the breakaway tab 54 is first completely detached from the blade carrier 42 and discarded. Once the tab 54 is removed, the mounting window 60 is no longer enclosed, which permits the insertion of the mounting portion 16 of the handle 4. In this way, the tab 54 also serves as an “unused” and/or “new blade” indicator. Removing the tab 54 “opens” the mounting window to receive the two prongs 18 and 20 of the handle 4. The configuration of the mounting window 60 provides a guide for the two prongs 18 and 20 of the handle during insertion/assembly of the handle 4.

The configuration of the window 60 and the prongs 18 and 20 also contribute to the audible “click” that ensures the user that the handle 4 is properly engaged with the blade assembly 10. Specifically, as the prongs 18 and 20 of the handle 4 enter the narrow channel defined by the intermediate portion of the mounting window 60, the outwardly biased, resilient prong 20 is squeezed closer to the rigid prong 18. Compressing the resilient prong 20 toward the rigid prong 18 in this manner puts pressure on the leading edge of the rigid prong's 18. As soon as rigid prong 18 advances past the shoulder 68 in the blade carrier 42, a clicking noise is heard as the leading edge snaps into place behind the shoulder 68. Once mounted to the handle, the locking tooth 22 on the rigid prong 18 is securely retained behind the shoulder 68 at the second end of the mounting window 60. The pressure exerted by the outwardly biased, resilient prong 20 on the rigid prong 18 ensures the continued engagement of the locking tooth 22 with the shoulder 66 while the scalpel is in use.

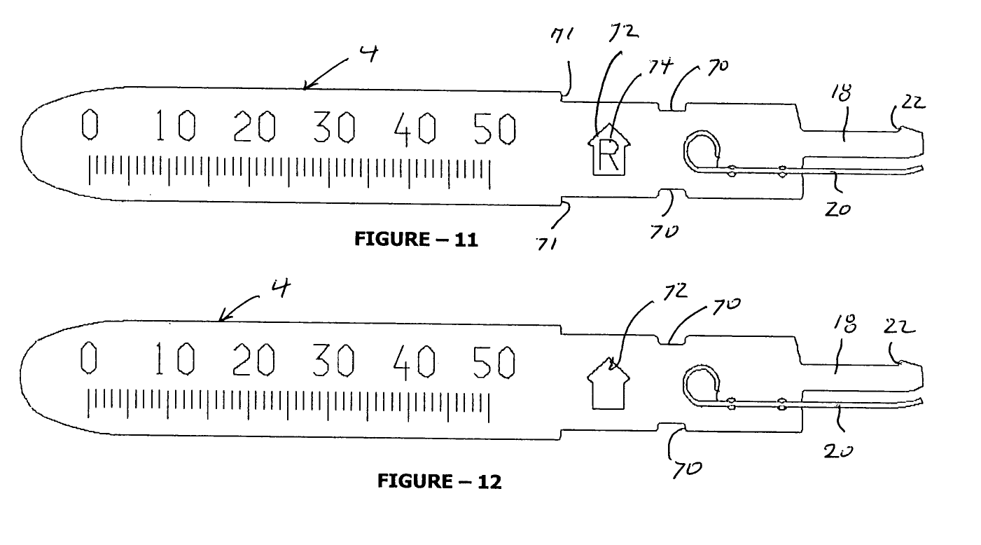

Referring especially to FIGS. 9a-e, the handle 4 has at least one locking notch 70 intermediate of the proximal and distal ends 6 and 8. The locking notch 70 is engaged by the dependant tooth 48 of the lever 44 on the protective sleeve 14 when the scalpel is in use. The handle 4 may also have a pair of shoulders 71, directly opposed to each other, that serve as “stop” surfaces that help to retain the protective sleeve 14 in a secure manner while the handle 4 is engaged with the blade assembly 10.

In another embodiment as shown in FIGS. 11 and 12, the handle 4 may have a pair of locking notches 70 directly opposed to each other, intermediate of the proximal and distal ends 6 and 8. With the configuration of the channel forming the mounting window being symmetrical, the blade carrier 42 is “orientation insensitive” when mounted to a handle 4 having a pair of opposed locking notches 68.

When the scalpel 2 is assembled with the handle 4 inserted into the blade assembly 10 as shown in FIGS. 9a and 9b, the locking tooth 22 on the rigid prong 18 is secured behind the shoulder 68 in the mounting window 60 of the blade carrier 42. At this point, the protective sleeve 14 is movable between two positions, a covered position and a cutting position. In the covered position as shown in FIG. 9b, the depending tooth 48 of the lever 44 on the protective sleeve 14 releasably engages the notch 52 in the blade carrier 42. While in this position, the protective sleeve 14 entirely covers the blade 12. In the second position, the cutting position as shown in FIG. 9c, the protective sleeve 14 is moved partially over the distal end 8 of the handle 4 and the depending tooth 48 engages the locking notch 70 in the handle 4, thereby exposing the blade 12 for use.

As shown in FIGS. 10a and 10b, to move the protective sleeve 14 between the two positions, the user must first press on the finger button 50 of the lever 44 which will raise the depending tooth 48 and disengage it from a respective locking notch 52 or 70. Once the tooth 48 has been disengaged, the protective sleeve 14 will slide easily over the surface of the handle 4. In this way, positioning the protective sleeve 14 between the covered position and the cutting position can be accomplished using only one hand as shown.

Referring to FIG. 9d and 9e, to safely dispose of the blade assembly 10 after use, the user first reestablishes the covered position by sliding the protective sleeve 14 over the blade 12 and engaging the dependent tooth 48 with the notch 52 on the blade carrier 42. Once the blade 12 is covered, the blade assembly 10 can be separated from the handle by bending the blade assembly upwardly as show in FIG. 9d until the locking tooth 22 on the rigid prong 18 of the handle disengages the shoulder 68 of the mounting window 60 in the blade assembly 10. It is preferred that the blade assembly 10 be bent toward the locking tooth 22, which will allow the locking tooth 22 to move past the shoulder 66. Once the locking tooth 22 is completely disengaged from the shoulder 66, the blade assembly 10 can then be pulled off the handle 4 as shown in FIG. 9e and safely discarded.

In another embodiment shown in FIGS. 11 and 12, the handle 4 may have a marking, such as an arrow 72, that points in the direction that the blade assembly 10 must be bent to disengage the blade assembly 10 from the handle 4. Preferably, the arrow 72 will be stamped in the surface of the handle 4. The arrow 72 may be stamped on one or both sides of the handle and may also include a letter 74 such as “R” for “removal” as shown in FIG. 10a. Alternatively, an arrow shape may be punched or cut out of the handle creating a hole in the handle as shown in FIG. 10b. It should be understood that the arrow (whether stamped or cut out) is visible only when the protective sleeve 14 is covering the blade, i.e. the covered condition.

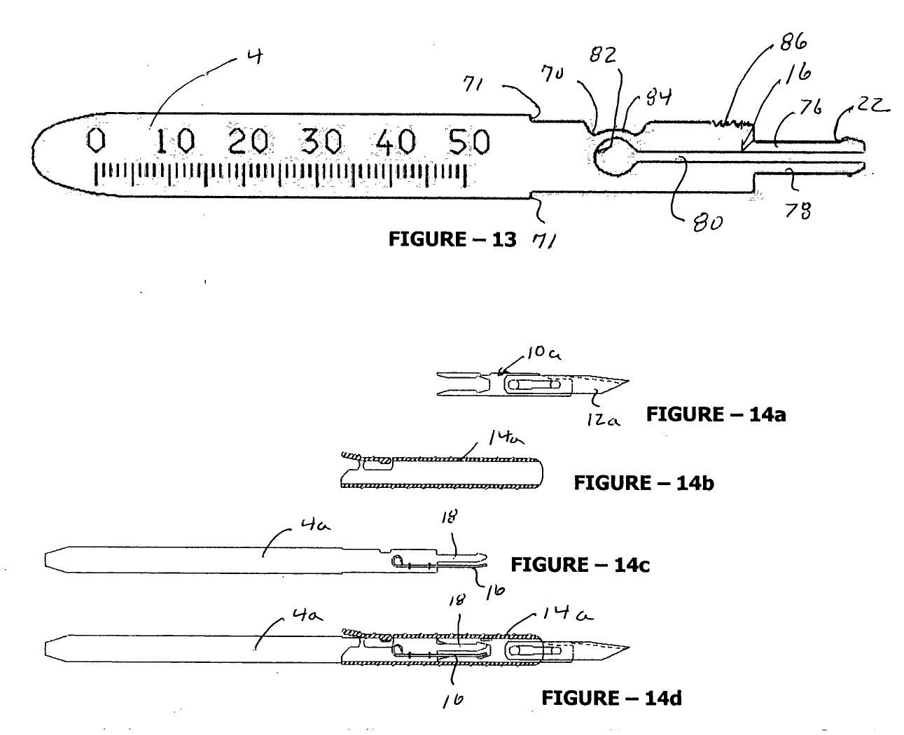

In another embodiment as shown in FIG. 13, the same retention/engagement system on the blade assembly 10 and protective sleeve 14 as previously described is used, but the mounting portion 16 of the handle 4 is modified. According to this embodiment, the blade assembly 10 may be removed by pushing on a portion of the mounting portion 10 of the handle 4. It should be understood that this embodiment does not require bending to release the blade assembly 10. According to this embodiment, the mounting portion 16 of the handle may have at least two extending prongs 76 and 78 that define a narrow channel 80 in-between. At least one of the prongs 76 has the locking tooth 22 at its end. It is preferred that the narrow channel 80 extends between the two prongs 76 and 78 toward the proximal end 6 of the handle and terminates in an enlarged substantially circular opening 82. Adjacent to the circular opening 82 is the locking notch 70 which has an arcuate bottom 84 as shown. The mounting portion 16 of the handle 4 of this embodiment also includes the pair of opposing shoulders 71 that act as stopping surfaces for the protective sleeve 14 on the blade carrier 42. The mounting portion 16 of the handle 4 removeably engages the mounting window 60 located on the blade carrier 42 after the removal of a breakaway tab 54 as described above. It is further preferred that the mounting portion 16 of the handle 4 makes an audible “clicking” noise when fully engaged with the blade assembly 42. Similar to the other embodiments described herein, the handle 4 is completely engaged with the blade assembly 10 when the leading edge of at least one of the prongs 76 or 78 makes an audible “click” as it advances past the shoulder 68 in the mounting window 16 in the blade carrier 42.

It is also preferred that the mounting portion 10 of the handle of the embodiment of FIG. 13 has a serrated or otherwise textured area 86 located opposite of at least one of the prongs 76 or 78. According to this embodiment, to separate the blade assembly 10 from the handle 4 when the protective sleeve 14 is in the covered position, the user preferably presses on the serrated or textured area 86 to disengage the locking tooth 22 from the shoulder 68. The serrated or otherwise textured surface area 86 allows the user to compress the prongs 76 and 78 together efficiently by providing a surface with increased traction under wet and/or slippery conditions. The serrated texture 86 also allows the user to locate the area and disengage the blade assembly without having to look down. It should be understood that the area 86 is not limited to having a serrated texture. Alternatively, the texture could be grooves, dots, etc. or combinations thereof. It is preferred that pressing on the serrated area squeezes the prongs 76 and 78 together (similar to a pair of tweezers) which facilitates the disengagement of the locking tooth 22 from the shoulder 68. Once the locking tooth 22 moves past the shoulder 68, the blade assembly 10 can be removed from the handle 4 and discarded without any bending of the blade assembly 10. In other words, the blade assembly 10 may be pulled straight off the handle in a substantially lateral direction.

Another preferred embodiment employs the same mounting principle but is miniaturized for use with a narrow surgical scalpel for specialized applications as illustrated in FIG. 11a-d. The handle 4a, blade assembly 10a and protective sleeve 14a all contain the same mounting and functioning elements as in the previous embodiments except that the individual components are miniaturized.

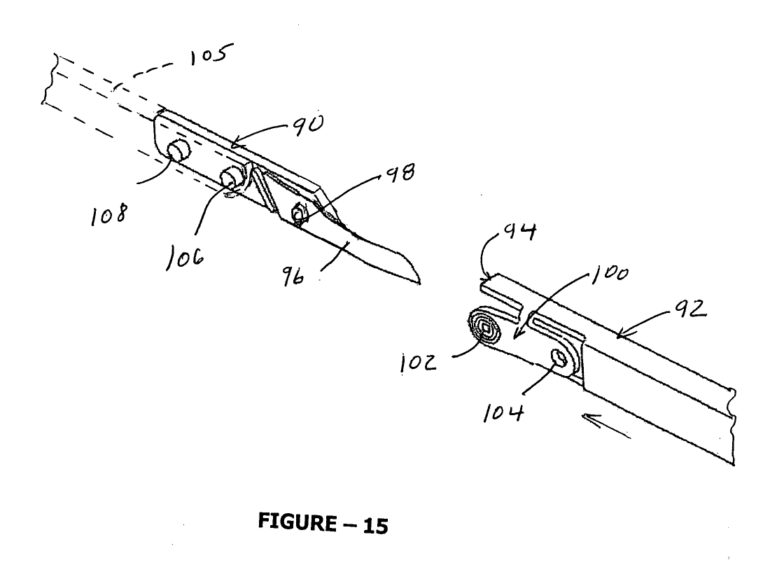

FIG. 15 shows another embodiment of a blade carrier 90 and protective sleeve 92. The protective sleeve 92 has an open end 94 to receive the blade carrier 90 which has a blade 96 extending from one end thereof. According to this embodiment, the blade carrier 90 is provided with at least one protrusion 98 in the form of a pin extending from the side of the blade carrier 90. The protective sleeve 92 includes a fulcrumed lever 100 pivotable on the side edge of the sleeve 92 as shown. The lever 100 has a finger portion 102 at its end closest to the open end 94 and a depression or opening 104 at the other end for releasably receiving the protrusion or pin 98 on the blade carrier 90.

To mount the protective sleeve 92 on the blade carrier 90, the blade carrier 90 is inserted into the open end 94 until the protrusion or pin 98 is engaged with the depression or opening 104 in the sleeve 92. At this point, the protective sleeve is held in a position covering the blade 96.

The blade carrier 90 may be provided with two additional pins 106 and 108 axially spaced from each other and spaced from the pin 98 in a direction away from the blade 96 as shown. It is to be understood that a handle (shown by dotted lines and indicated by the reference number 105) may be attached to the blade carrier 90 by virtue of openings on the handle 105 through which the pins 106 and 108 extend.

When it is desired to move the protective sleeve 92 relative to the blade carrier 90, the finger portion 102 of the fulcrumed lever 100 may be depressed. This causes the depression or opening 104 at the other end of the fulcrumed lever 100 to disengage from the protrusion or pin 98 on the blade carrier 90 and permit the protective sleeve 92 to be moved relative to the blade carrier 90. When the handle 105 is mounted on the blade carrier 90, the protective sleeve 92 may be moved toward the handle 105 until the opening or depression 104 engages the pin 106. In this position, the protective sleeve 92 still covers the blade 96 and maintains the handle 105 in engagement with the blade carrier 90.

The third protrusion or pin 108 provides a locking arrangement for the protective sleeve 92 when the sleeve 92 is moved into its rearward position in which the blade 96 is exposed for cutting. In this position, the depression or opening 104 in the lever 100 releasably engages the protrusion or pin 108 holding the protective cover 92 in the cutting position.

Those familiar with this field of endeavor will appreciate that one or more of advantages of the preferred embodiments include: (1) increased assurance that the blade assembly is properly engaged with the handle; (2) simplified sleeve and handle retention features permit an “orientation insensitive” assembly of the scalpel; and (3) effective prevention against the accidental reuse of a contaminated blade.

While various modifications have been shown and described, various modifications and substitutions may be made thereto. Accordingly, it is understood that the present embodiments have been described by way of illustration and not limitation.

1. A blade assembly for a surgical scalpel comprising:
a blade carrier;
a blade attached to said blade carrier at one end thereof;
a protective cover covering said blade and blade carrier;
said protective cover having a portion in releasable engagement with said blade carrier to hold said protective cover in a protecting position covering said blade carrier and said blade.

2. The blade assembly of claim 1, wherein said blade carrier includes a tab portion at its end opposite said blade, said tab portion having a shoulder thereon adapted to be engaged by said protective cover upon movement of said protective cover in a direction toward the end of said blade carrier opposite to said blade.

3. The blade assembly of claim 2, wherein said tab portion is designed to be broken away from the remainder of said blade carrier.

4. The blade assembly of claim 1, wherein said blade carrier has a notch and said protective cover has tooth in releasable engagement with said notch.

5. The blade assembly of claim 4, wherein said tooth on said protective cover is attached to one end of a fulcrumed lever and said other end of said lever has a finger engaging portion.

6. The blade assembly of claim 5, wherein said fulcrumed lever includes resilient legs intermediate said tooth and said finger engaging portion attached to said protective cover whereby depressing said finger engaging portion will raise said tooth.

7. The blade assembly of claim 1, wherein said blade carrier has a protrusion and said protective cover has a depression for releasably engaging said protrusion.

8. The blade assembly of claim 7, wherein said depression is on one end of a fulcrumed lever, the other end of said lever having a finger engaging portion.

9. The blade assembly of claim 8, wherein said protrusion is a pin and said depression is an opening adapted to releasably receive said pin.

10. The blade assembly of claim 8, wherein said blade assembly includes two spaced pins, said depression on said lever engaging one of said pins when said protective cover covers said blade and said depression engaging said the other of said pins when said blade is exposed.

11. A surgical scalpel comprising:
a handle having two prongs extending from one end thereof, one of said prongs being resilient, the other of said prongs having a latching tooth at its distal end;
a disposable blade assembly including a blade carrier having a surgical blade attached thereto, said blade carrier having a surface defining a window with a shoulder into which said prongs extend with said latching tooth engaging said shoulder when said handle is inserted into said blade carrier.

12. The surgical scalpel of claim 11, wherein said resilient prong engages said surface to maintain said latching tooth in engagement with said shoulder.

13. The surgical scalpel of claim 11, wherein said resilient prong is a leaf spring.

14. The surgical scalpel of claim 13, wherein said leaf spring is mounted in a slot opening into the handle, said leaf spring being staked in position.

15. The surgical scalpel of claim 11, wherein said prongs are both integral with said handle.

16. The surgical scalpel of claim 15, wherein said handle includes a stainless steel insert having said prongs extending therefrom and a plastic over-coating covering said insert with the prongs extending therefrom.

17. The surgical scalpel of claim 11, wherein said window includes a narrowing ramp at its opening and said resilient prong includes a ramp portion which engages said narrowing ramp upon insertion of said handle into said blade assembly.

18. The surgical scalpel of claim 11, including a protective cover on said blade assembly, said protective cover being movable between a covered position wherein it covers said blade and a cutting position wherein the blade is exposed for use.

19. The surgical scalpel of claim 18, further including releasable latching means between said protective cover and said blade carrier to releasably maintain said protective cover in said covered position and between said protective blade carrier and said handle to releasably maintain said protective cover in said cutting position.

20. The surgical scalpel of claim 18, wherein said blade carrier has a notch and said protective cover has a tooth in releasable engagement with said notch to releasably maintain said protective cover in said covered position.

21. The surgical scalpel of claim 10, wherein said handle has a notch, said notch in said handle receiving said tooth when said protective cover is moved into its cutting position.

22. The surgical scalpel of claim 21, wherein said tooth on said protective cover is attached to one end of a fulcrumed lever which is connect to said protective cover, said other end of said lever having a finger engaging portion.

23. The surgical scalpel of claim 22, wherein said fulcrumed lever includes resilient legs intermediate said latch and said finger engaging portion attached to said protective cover whereby depressing said finger engaging portion will raise said latch potion.

24. The surgical scalpel of claim 21, wherein said handle has two oppositely disposed notches.

25. A surgical scalpel comprising:
a handle having two prongs extending from one end thereof, one of said prongs having a latching tooth at its distal end, said prongs being separated by a channel extending into said handle; and
a disposable blade assembly including a blade carrier having a surgical blade attached thereto, said blade carrier having a surface defining a window with a shoulder into which said prongs extend with said latching tooth engaging said shoulder when said handle is inserted into said blade carrier.

26. The surgical scalpel of claim 25, wherein said handle includes a textured area opposite said prong having said latching tooth for engagement by a user to press to disengage the latching tooth from said shoulder to enable said blade assembly to be removed from said handle.

27. A surgical scalpel comprising:
a disposable blade assembly having a first end and a second end defining at least one end of a mounting window;
a protective sleeve on said blade assembly, and
a reusable handle having a distal end and a proximal end defining a mounting portion that releasably engages said mounting window;
wherein said mounting portion engages said mounting window with an audible click that ensures complete engagement.

28 The surgical scalpel of claim 27, wherein
said mounting portion of said reusable handle further comprises at least two prongs and a locking tooth; and
said at least two prongs and said locking tooth releasably engage said disposable blade assembly.
